(19) United States
(12) Patent Application Publication
(10) Pub. No.: US 2023/0320696 A1
(43) Pub. Date: Oct. 12, 2023

(54) ULTRASONIC ENDOSCOPE AND METHOD FOR ASSEMBLING ULTRASONIC ENDOSCOPE

(71) Applicant: FUJIFILM Corporation, Tokyo (JP)

(72) Inventor: Yuto SHIMOYAMA, Kanagawa (JP)

(73) Assignee: FUJIFILM Corporation, Tokyo (JP)

(21) Appl. No.: 18/336,023

(22) Filed: Jun. 16, 2023

Related U.S. Application Data
(63) Continuation of application No. PCT/JP2021/047235, filed on Dec. 21, 2021.

(30) Foreign Application Priority Data
Jan. 12, 2021 (JP) 2021-002651

(51) Int. Cl. A61B 8/00; A61B 8/12

(52) U.S. Cl. CPC A61B 8/445; A61B 8/12

(57) ABSTRACT

An ultrasonic endoscope (1) including a distal end body block component (260) in which an ultrasound transducer (50), an observation optical system (40), and an illumination optical system (44) are mounted, and a channel block component (70) in which a channel into which a treatment tool is inserted is mounted, in which the ultrasonic endoscope (1) has a first load receiving structure (110) in which the channel block component (70) receives a load from the distal end body block component (260), and a second load receiving structure (120) in which the distal end body block component (260) receives a load from the channel block component (70).

### CROSS-REFERENCE TO RELATED APPLICATIONS

The present application is a Continuation of PCT International Application No. PCT/JP2021/047235 filed on Dec. 21, 2021 claiming priority under 35 U.S.C § 119(a) to Japanese Patent Application No. 2021-002651 filed on Jan. 12, 2021. Each of the above applications is hereby expressly incorporated by reference, in its entirety, into the present application.

### BACKGROUND OF THE INVENTION

### 1. Field of the Invention

The present invention relates to an ultrasonic endoscope and a method for assembling an ultrasonic endoscope, and in particular, to an ultrasonic endoscope having an outlet port from which a treatment tool is led out, in a distal end part of an insertion part, and a method for assembling an ultrasonic endoscope.

### 2. Description of the Related Art

As an ultrasonic endoscope, an ultrasonic endoscope that comprises an electronic scanning type ultrasound oscillator in a distal end part of an insertion part of an endoscope is known. The ultrasonic endoscope uses a treatment tool, such as a puncture needle, led out from an outlet port of the distal end part through a treatment tool insertion channel and inserted into a lesion part to collect a cellular tissue of a lesion part, while acquiring an ultrasound image of the lesion part using the ultrasound oscillator.

The ultrasonic endoscope comprises an observation optical system and an illumination optical system in addition to the ultrasound oscillator, can also perform observation via an optical image, and can perform observation via the optical image until the puncture needle is made to approach and is punctured into a body wall, to reliably guide the puncture needle to a target site.

As such an ultrasonic endoscope, for example, JP2004-135937A described below describes an ultrasonic endoscope in which an endoscope observation part and an ultrasound transducer are mounted in a distal end hard part of an insertion part, and a treatment tool channel is opened between the endoscope observation part and the ultrasound transducer. JP1999-276422A (JP-H11-276422A) describes an ultrasonic endoscope in which an ultrasound examination mechanism and an endoscopic observation mechanism are provided in a distal end constituent part of an insertion part, and a treatment tool outlet part from which a treatment tool is led out is provided between the ultrasound examination mechanism and the endoscopic observation mechanism.

### SUMMARY OF THE INVENTION

The ultrasonic endoscope is required to secure the insulation of the distal end part as an ultrasound safety standard. For this reason, a distal end part body is formed of a resin component, and improvement of strength and durability of the distal end part is required. In particular, in an ultrasonic endoscope that is applied to a bronchus, a reduction in distal end diameter is required, and there is a limit to improvement of strength and durability by increasing the thickness of the component.

In the ultrasonic endoscope, since the puncture needle has stiffness, force received when the puncture needle is punctured into a living body tissue is applied to a component that holds the treatment tool outlet port, force received when the ultrasound oscillator is brought into contact with a bronchial wall surface is applied to a component that holds the ultrasound oscillator, and the directions of loads are different, which requires strength and durability to such an extent that the distal end part is not broken by any load.

In the configuration of the distal end part, it is required to reduce repairability costs by providing a component with an ultrasound oscillator cable or an observation optical system, which are high-cost parts, and a component with the outlet port, which is a high-frequency replacement part, as separate members and having a structure capable of being assembled and disassembled.

In the ultrasonic endoscope described in JP2004-135937A, while a distal end component is divided into two parts, since the distal end component is divided into two upper and low components, there is a problem in that strength is weak with respect to a load in a peeling direction applied from an endoscope distal end. While the distal end component is fixed by screws, because of residual stress of the resin component and the need for a shape of a rib or the like to be a screw tap shape, a problem occurs in terms of a reduction in size of the distal end part.

In the ultrasonic endoscope described in JP1999-276422A (JP-H11-276422A), since the ultrasound oscillator, the observation optical system, the illumination optical system, and the treatment tool channel are attached to the integrated distal end component, the component is less likely to be peeled even though a load is applied to the distal end part. However, since the entire distal end component is formed integrally, even though the treatment tool outlet part is deteriorated due to reaction force of the puncture needle, the entire distal end component needs to be replaced, and a problem occurs in terms of repairability.

The present invention has been accomplished in view of such a situation, and an object of the present invention is to provide an ultrasonic endoscope and a method for assembling an ultrasonic endoscope that disperses reaction force with respect to a load given to a distal end part of an endoscope to components configuring the distal end part to ensure strength and durability and to improve repairability.

To attain the object of the present invention, there is provided an ultrasonic endoscope according to the present invention comprising an ultrasound transducer in a distal end part, a distal end body block component in which an ultrasound transducer, an observation optical system, and an illumination optical system are mounted, and a channel block component in which a channel into which a treatment tool is inserted is mounted, in which the ultrasonic endoscope has a first load receiving structure that has a first supported surface provided in the distal end body block component and a first support surface provided in the channel block component to face the first supported surface, and in which the first support surface supports the first supported surface, such that the channel block component receives a load from the distal end body block component, and a second load receiving structure that has a second supported surface provided in the channel block component and a second support surface provided in the distal end body block component to face the second supported surface, and in which the second support surface supports the second supported surface, such that the distal end body block component receives a load from the channel block component.

According to an aspect of the present invention, it is preferable that the distal end body block component includes an ultrasound block component in which the first supported surface is provided and the ultrasound transducer is mounted, and an optical system block component in which the second support surface is provided and the observation optical system and the illumination optical system are mounted, and the ultrasonic endoscope has a third load receiving structure that has a third supported surface provided in the optical system block component and a third support surface provided in the ultrasound block component to face the third supported surface, and in which the third support surface supports the third supported surface, such that the ultrasound block component receives a load from the optical system block component.

According to an aspect of the present invention, it is preferable that the third supported surface and the third support surface are perpendicular to a scanning surface of the ultrasound transducer and are parallel to a surface perpendicular to a longitudinal axis direction of the distal end part.

According to an aspect of the present invention, it is preferable that at least one of the third supported surface or the third support surface has a seal material filling groove portion.

According to an aspect of the present invention, it is preferable that the optical system block component has a first guide portion along which the channel block component is slidable and disposable.

According to an aspect of the present invention, it is preferable that the ultrasound block component has a second guide portion along which the optical system block component is slidable and disposable.

According to an aspect of the present invention, it is preferable that a forming material of the distal end body block component is resin, and a forming material of the channel block component is metal.

According to an aspect of the present invention, it is preferable that the second supported surface is configured with a pair of flange surfaces that spread outward from both side surfaces of the channel block component opposite to each other.

According to an aspect of the present invention, it is preferable that the distal end body block component has an engaged portion in which the first supported surface is provided, and the channel block component has an engagement portion in which the first support surface is provided and which is engageable with the engaged portion, and the engaged portion and the engagement portion are engaged with each other, such that the channel block component is assembled to the distal end body block component.

According to an aspect of the present invention, it is preferable that a locking portion is provided in any one of the engaged portion or the engagement portion, and a locked portion that is locked to the locking portion to restrict sliding of the engagement portion with respect to the engaged portion is provided in the other portion.

To attain the object of the present invention, there is provided a method for assembling an ultrasonic endoscope including an ultrasound transducer in a distal end part according to the present invention, the method comprising a first load receiving structure forming step of forming a first load receiving structure having a structure in which a distal end body block component in which the ultrasound transducer, an observation optical system, and an illumination optical system are mounted is supported by a channel block component in which a channel into which a treatment tool is inserted is mounted, and a second load receiving structure forming step of forming a second load receiving structure having a structure in which the channel block component is supported by the distal end body block component.

According to an aspect of the present invention, it is preferable that the distal end body block component includes an ultrasound block component in which the ultrasound transducer is mounted, and an optical system block component in which the observation optical system and the illumination optical system are mounted, the first load receiving structure is a structure in which the ultrasound block component is supported by the channel block component, the second load receiving structure is a structure in which the channel block component is supported by the optical system block component, and the method for assembling an ultrasonic endoscope further comprises a third load receiving structure forming step of forming a third load receiving structure having a structure in which the optical system block component is supported by the ultrasound block component.

According to an aspect of the present invention, it is preferable that the first load receiving structure forming step and the third load receiving structure forming step are performed after the second load receiving structure forming step is performed.

According to the present invention, it is possible to disperse a load applied to the distal end part of the ultrasonic endoscope to components configuring the distal end part, and to ensure strength and durability of the distal end part. It is also possible to reduce repair costs in breakage.

### BRIEF DESCRIPTION OF THE DRAWINGS

FIG. 1 is a general view of an ultrasonic endoscope.

FIG. 2 is a perspective view of a distal end hard part.

FIG. 3 is an exploded perspective view of the distal end hard part.

FIG. 4 is a sectional view of the distal end hard part.

FIG. 5 is a sectional view of the distal end hard part and is an enlarged view of a first load receiving structure.

FIG. 6 is a sectional view of the distal end hard part taken along line VI-VI of FIG. 2.

FIG. 7 is a perspective view from a cross section side taken along the line VI-VI of FIG. 2.

FIG. 8 is a diagram illustrating a method for assembling an endoscope.

FIG. 9 is a diagram illustrating the method for assembling an endoscope.

FIG. 10 is a diagram illustrating the method for assembling an endoscope.

FIG. 11 is a perspective view of a distal end hard part of another embodiment.

FIG. 12 is an exploded perspective view of the distal end hard part shown in FIG. 11.

### DESCRIPTION OF THE PREFERRED EMBODIMENTS

Hereinafter, an ultrasonic endoscope and a method for assembling an ultrasonic endoscope according to the present invention will be described with reference to the accompanying drawings.

[Overall Configuration of Ultrasonic Endoscope]

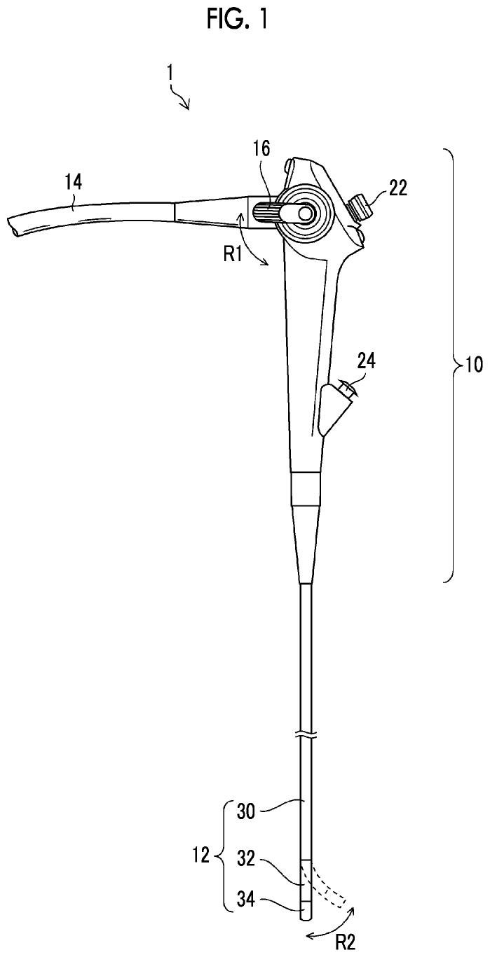

FIG. 1 is a general view of an ultrasonic endoscope 1. As shown in FIG. 1, the ultrasonic endoscope 1 (hereinafter, simply abbreviated as an “endoscope 1”) is configured with an operating part 10 that is gripped by a practitioner to perform various operations, an insertion part 12 that is inserted into a body cavity of a patient, and a universal cord 14. The endoscope 1 is connected to system constituent devices that configure an endoscope system, such as a processor device and a light source device (not shown), through the universal cord 14.

The operating part 10 is provided with various operation members that are operated by the practitioner. For example, an angle lever 16, a suction button 22, and the like of which the operations will be appropriately described below are provided.

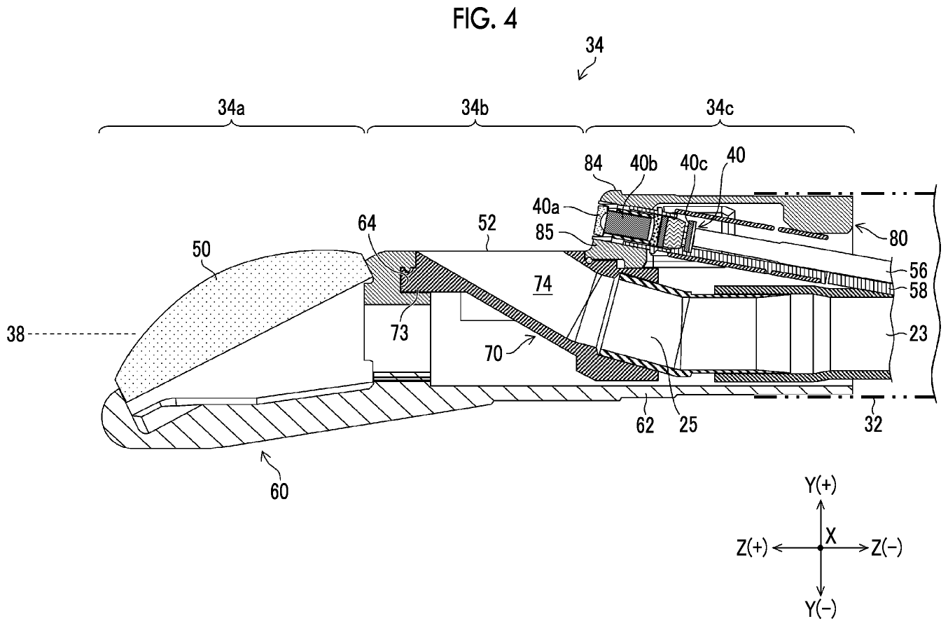

The operating part 10 is provided with a treatment tool inlet port 24 through which a treatment tool is inserted into a treatment tool insertion channel 23 (see FIG. 4) that is inserted into the insertion part 12.

The insertion part 12 extends from a distal end of the operating part 10 and is formed in a small-diameter elongated shape as a whole. The insertion part 12 is configured with, in order from a proximal end side toward a distal end side, a soft part 30, a bendable part 32, and a distal end hard part 34 as a distal end part.

The soft part 30 occupies most of the insertion part 12 from the proximal end side and has enough flexibility to be bent in any direction. In a case where the insertion part 12 is inserted into a body cavity, the soft part 30 is bent along an insertion path into the body cavity.

The bendable part 32 is bent in an up-down direction (R2 direction) by rotating the angle lever 16 of the operating part 10 in an R1 direction. With the bending operation of the bendable part 32, the distal end hard part 34 can be directed in a desired direction.

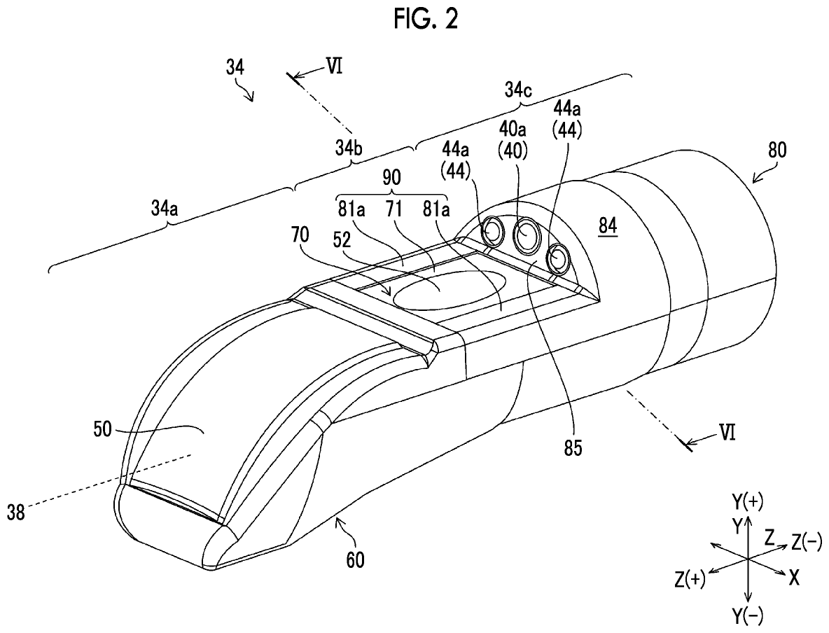

As will be described in detail below with reference to FIGS. 2 to 4, the distal end hard part 34 comprises an observation optical system 40 and illumination optical systems 44 that are provided to capture an observation image in the body cavity, an ultrasound transducer 50 that acquires an ultrasound image, and an outlet port 52 from which the treatment tool inserted from the treatment tool inlet port 24 is led out.

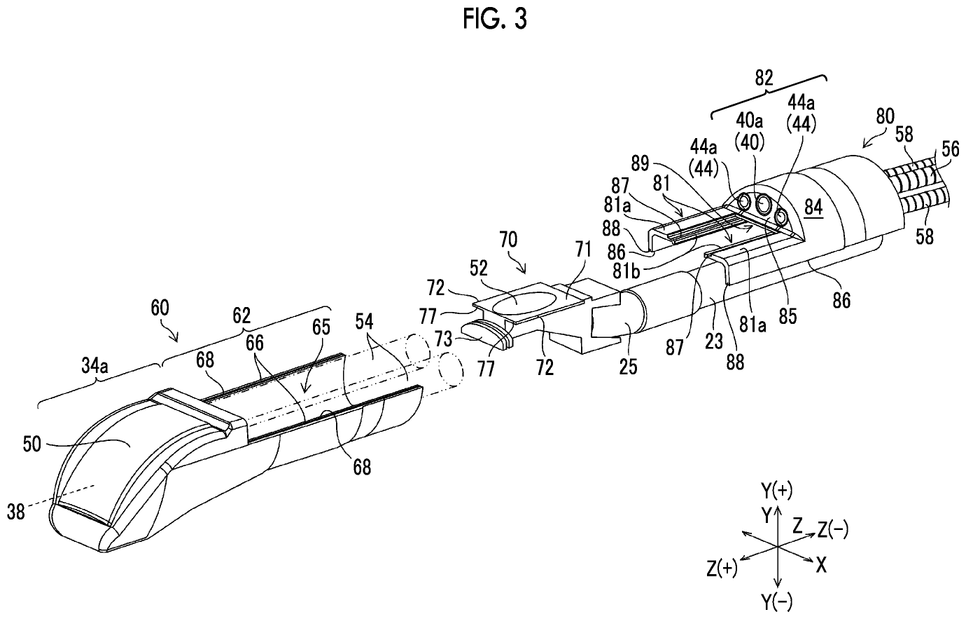

The universal cord 14 includes signal cables 54, a signal cable 56, and light guides 58 shown in FIGS. 3 and 4 described below in detail. A connector is provided in an end portion (not shown) of the universal cord 14. The connector is connected to predetermined system constituent devices that configure the endoscope system, such as a processor device and a light source device. As a result, power, control signals, illumination light, and the like necessary for the operation of the endoscope 1 are supplied from the system constituent devices to the endoscope 1. Conversely, data of the observation image acquired by the observation optical system 40 and data of the ultrasound image acquired by the ultrasound transducer 50 are transmitted from the endoscope 1 to the system constituent devices. The observation image and the ultrasound image transmitted to the system constituent devices are displayed on a monitor, and the practitioner or the like can observe the images.

The configuration of the operating part 10 is not limited to the aspect shown in FIG. 1. A pair of angle knobs may be provided instead of the angle lever 16, and the bendable part 32 may be bent in the up-down direction and in a right-left direction by rotating a pair of angle knobs. An air/water supply button may be provided in the operating part 10, and gas, such as air, a liquid for cleaning, and the like may be supplied to the distal end hard part 34 by operating the air/water supply button.

[Configuration of Distal End Hard Part]

FIG. 2 is a perspective view of the distal end hard part 34. FIG. 3 is an exploded perspective view of the distal end hard part 34. FIG. 4 is a sectional view of the distal end hard part 34.

A Z direction in the drawing is a direction parallel to a longitudinal axis 38 of the distal end hard part 34 (insertion part 12). A Z(+) direction side of the Z direction in the drawing is a distal end side of the distal end hard part 34, and a Z(−) direction side is a proximal end side of the distal end hard part 34. A Y direction in the drawing is a direction perpendicular to the Z direction and is an up-down direction in each drawing in the present embodiment. A Y(+) direction side as a one direction side of the Y direction is an up direction in the drawing, and a Y(−) direction side as the other direction side of the Y direction is a down direction in the drawing. An X direction in the drawing is a direction perpendicular to both the Z direction and the Y direction.

As shown in FIGS. 2 to 4, the distal end hard part 34 is configured by combining an ultrasound block component 60, a channel block component 70, and an optical system block component 80 (in particular, see FIG. 3). The distal end hard part 34 comprises an ultrasonic attachment part 34a, an outlet port forming part 34b, and a body part 34c from the distal end side toward the proximal end side of the distal end hard part 34 in a state in which the respective block components are combined (see FIGS. 2 and 4).

A forming material of the ultrasound block component 60 is an insulating material having insulation, and the ultrasound block component 60 is formed of a resin material, for example, plastic, such as polysulphone and polyether imide. The ultrasound block component 60 comprises the ultrasonic attachment part 34a and an optical system block component attachment part 62 from a distal end side toward a proximal end side thereof. The ultrasonic attachment part 34a and the optical system block component attachment part 62 are formed integrally.

The ultrasound transducer 50 is attached to the ultrasonic attachment part 34a in a posture tilted forward (inclined) to the Y(−) direction side with respect to the longitudinal axis 38 in a case of being viewed from the X direction side. The ultrasound transducer 50 is a convex type that has an ultrasonic wave transmitting and receiving surface on which ultrasound oscillators that transmit and receive ultrasonic waves are arranged in a curved shape along a direction of the longitudinal axis 38. Data for generating an ultrasound image of a part to be observed is acquired by the ultrasound transducer 50. The number of ultrasound oscillators that configure the ultrasound transducer 50 is not limited.

The optical system block component attachment part 62 extends from a region on the Y(−) direction side of the proximal end part of the ultrasonic attachment part 34a toward the proximal end side [Z(−) direction side] in a case where the distal end hard part 34 is viewed from the X direction side. An engaged portion 64 with which an engagement portion 73 of the channel block component 70 described below is engaged is formed in a region on the Y(+) direction side of the proximal end part of the ultrasonic attachment part 34a.

The optical system block component attachment part 62 has a substantially semi-cylindrical shape corresponding to a divided part on the Y(−) direction side (a lower half side) out of two divided parts obtained by dividing the outlet port forming part 34b and the body part 34c into two parts in the Y direction (into two parts vertically). For this reason, the optical system block component attachment part 62 has an attachment part opening 65 that is opened on the Y(+) direction side.

The attachment part opening 65 is formed parallel to an XZ plane and along the Z direction. Inside the attachment part opening 65 of the optical system block component attachment part 62, the signal cables 54 that connect the ultrasound transducer 50 and the system constituent devices described above are disposed.

In the optical system block component attachment part 62, a pair of guide portions 66 that forms the attachment part opening 65 is formed, and the pair of guide portions 66 extends to the Z(−) direction side along the attachment part opening 65. The pair of guide portions 66 are formed with a surface perpendicular to an ultrasonic wave transmitting and receiving surface (corresponding to a “scanning surface of an ultrasound transducer” of the present invention) and parallel to a surface perpendicular to the direction of the longitudinal axis 38 of the distal end part. The optical system block component 80 described below is attached to the pair of guide portions 66 while being slid in the Z direction. As a result, the optical system block component 80 is attached to the optical system block component attachment part 62, that is, the ultrasound block component 60, through the pair of guide portions 66.

With the configuration of the pair of guide portions 66, the optical system block component attachment part 62 of the ultrasound block component 60 can be made to have a semicircular shape. The shape of the optical system block component attachment part 62 is made to be a semicircular shape, whereby it is possible to easily perform molding since a release direction of a mold can be set to only the Y direction in resin-molding the optical system block component attachment part 62.

The pair of guide portions 66 is provided with seal material filling groove portions 68 to secure airtightness of connection surfaces to the optical system block component 80. The groove portions 68 are filled with a seal material, and the optical system block component 80 is attached to the groove portions 68, whereby it is possible to secure airtightness of the inside of the distal end hard part 34. In a case where groove portions 88 are provided in a pair of guided portions 86 of the optical system block component 80 described below to be matching surfaces of the pair of guide portions 66, the groove portions 68 may not be provided in the pair of guide portions 66.

The channel block component 70 configures the outlet port forming part 34b along with the optical system block component 80, and a forming material of the channel block component 70 is metal. As metal, a known metal material can be used. The channel block component 70 has the outlet port 52 of the treatment tool that is opened on the Y(+) direction side, and a substantially rectangular opening forming surface 71 parallel to the XZ plane where the outlet port 52 is opened and along the Z direction (including the longitudinal axis 38; the same applies hereinafter).

In both end portions in the X direction of the opening forming surface 71 of the channel block component 70, a pair of flange surfaces 72 parallel to the XZ plane is formed along the Z direction (see FIG. 3). The pair of flange surfaces 72 is used for attachment of the channel block component 70 to the optical system block component 80, and extends outward (X direction) from both side surfaces in the X direction of the opening forming surface 71.

The engagement portion 73 that is engageable with the engaged portion 64 of the ultrasonic attachment part 34a is formed on a distal end side of the channel block component 70.

An in-block pipe line 74 is formed inside the channel block component 70. A distal end side of the in-block pipe line 74 is connected to the outlet port 52, and a proximal end side of the in-block pipe line 74 is connected to the treatment tool insertion channel 23 inserted into the insertion part 12, through a channel connection pipe 25. As a result, a distal end of the treatment tool inserted from the treatment tool inlet port 24 is guided to the outlet port 52 by way of the treatment tool insertion channel 23, the channel connection pipe 25, and the in-block pipe line 74, and is led out from the outlet port 52 to the outside.

The optical system block component 80 is formed of a resin material, like the ultrasound block component 60. The optical system block component 80 has a shape corresponding to a divided part on the Y(+) direction side (an upper half side) out of the two divided parts obtained by dividing the outlet port forming part 34b and the body part 34c into two parts in the Y direction (into two parts vertically).

The optical system block component 80 comprises, from a distal end side toward a proximal end side thereof, a pair of channel block component attachment portions 81 that is provided at an interval in the X direction, and an optical system storage portion 82 (see FIG. 3). The pair of channel block component attachment portions 81 and the optical system storage portion 82 are formed integrally.

The pair of channel block component attachment portions 81 extends from positions [positions on the Y(−) direction side] slightly lower than an apex on the Y(+) direction side of the optical system storage portion 82 with respect to the apex to a distal end side [Z(+) direction side] of the optical system storage portion 82 in a case where the optical system block component 80 is viewed from the X direction side.

A space for attaching the channel block component 70 is secured between the pair of channel block component attachment portions 81. In end portions on the Y(+) direction side of the pair of channel block component attachment portions 81, a pair of planes 81a having a shape parallel to the XZ plane and along the Z direction is formed. A pair of support surfaces 81b is formed at positions shifted from the pair of planes 81a in end portions to the above-described space side on the Y(−) direction side of the pair of channel block component attachment portions 81.

The pair of support surfaces 81b has a shape parallel to the XZ plane and along the Z direction, and is formed at positions on the Y(−) direction side slightly lower than the pair of planes 81a by the amount of thickness in the Y direction of the pair of flange surfaces 72. The pair of support surfaces 81b supports the pair of flange surfaces 72 from both sides in the X direction. As a result, through the pair of flange surfaces 72 and the pair of support surfaces 81b, the channel block component 70 is supported to be slidable in the Z direction between the pair of channel block component attachment portions 81. As a result, the channel block component 70 can be attached to the optical system block component 80 while sliding in the Z direction. Then, the channel block component 70 is adhered and assembled to the optical system block component 80. Groove portions 77 and 87 for an adhesive to which an adhesive is applied are provided at positions facing the pair of flange surfaces 72 and the pair of support surfaces 81b.

In a case where the channel block component 70 is attached to the optical system block component 80, the opening forming surface 71 and the pair of planes 81a form a continuous plane 90. The continuous plane 90 is a plane parallel to the XZ plane and along the Z direction, and configures a part of an outer peripheral surface of the distal end hard part 34.

The optical system storage portion 82 has a semi-cylindrical shape, and has a convex surface 84 and a stepped surface 85. The convex surface 84 configures a part of the outer peripheral surface of the distal end hard part 34. The convex surface 84 is a surface that configures an outer peripheral surface of the optical system storage portion 82, and is a surface that is positioned on the Y(+) direction side with respect to the continuous plane 90 and that has a shape along the Z direction. In the optical system storage portion 82, a pair of guided portions 86 that extends in the Z(−) direction for forming a storage opening portion 89 opened in the Y(−) direction is formed. The pair of guided portions 86 are portions that are matching surfaces of the pair of guide portions 66 in assembling the distal end hard part 34. Accordingly, the guided portions 86 are formed with a surface perpendicular to the ultrasonic wave transmitting and receiving surface and parallel to a surface perpendicular to the direction of the longitudinal axis 38 of the distal end part.

With the configuration of the pair of guided portions 86, the optical system storage portion 82 of the optical system block component 80 can be made to have a semicircular shape. The shape of the optical system storage portion 82 is made to be a semicircular shape, whereby it is possible to easily perform molding since a release direction of a mold can be set to only the Y direction in resin-molding the optical system storage portion 82.

The pair of guided portions 86 is provided with seal material filling groove portions 88 to secure airtightness of connection surfaces to the ultrasound block component 60. The groove portions 88 are filled with a seal material, and the ultrasound block component 60 is attached to the groove portions 88, whereby it is possible to secure airtightness of the inside of the distal end hard part 34. In a case where the groove portions 68 are provided in the pair of guide portions 66, the groove portions 88 may not be provided.

The stepped surface 85 is an inclined surface that connects a proximal end side of the continuous plane 90 and a distal end side of the convex surface 84, and configures a part of the outer peripheral surface of the distal end hard part 34. The inclined surface used herein includes a vertical surface having an angle of 90° with respect to the Z direction.

The stepped surface 85 is provided with an observation window 40a of the observation optical system 40 and illumination windows 44a of a pair of illumination optical systems 44.

The observation optical system 40 includes an observation window 40a provided in the stepped surface 85, and a lens system 40b and a charge-coupled device (CCD) type or a complementary metal oxide semiconductor (CMOS) type imaging element 40c provided in the optical system storage portion 82. The imaging element 40c captures an observation image fetched from the observation window 40a through the lens system 40b. Then, the imaging element 40c outputs an imaging signal of the observation image to the system constituent devices through the signal cable 56 inserted into the insertion part 12.

The illumination optical systems 44 are provided on both sides of the observation optical system 40 in the X direction, and each of the illumination optical systems 44 includes the illumination window 44a provided in the stepped surface 85, and the light guide 58 inserted into the insertion part 12. An emission end of the light guide 58 is disposed rearward of each illumination window 44a. As a result, illumination light supplied from the light source device to each light guide 58 is emitted from each illumination window 44a.

In a case where the channel block component 70 is attached to the optical system block component 80, the pair of guided portions 86 is attached to the optical system block component attachment part 62 of the ultrasound block component 60 through the pair of guide portions 66.

As described above, the ultrasound block component 60, the channel block component 70, and the optical system block component 80 are combined, and the distal end hard part 34 is formed. As a result, in a case where the distal end hard part 34 is viewed from the Y(+) direction side (upper side), the ultrasound transducer 50, the outlet port 52, and the stepped surface 85 (observation window 40a) are disposed in order from the distal end side toward the proximal end side of the distal end hard part 34.

<Dispersion Structure of Load>

In the distal end hard part 34 of the present embodiment, a first load receiving structure 110 in which the channel block component 70 receives a load from the ultrasound block component 60 is provided. Furthermore, a second load receiving structure 120 in which the optical system block component 80 receives a load from the channel block component 70 is provided. In addition, a third load receiving structure 130 in which the ultrasound block component 60 receives a load from the optical system block component 80 is provided. Hereinafter, each load receiving structure will be described.

(First Load Receiving Structure)

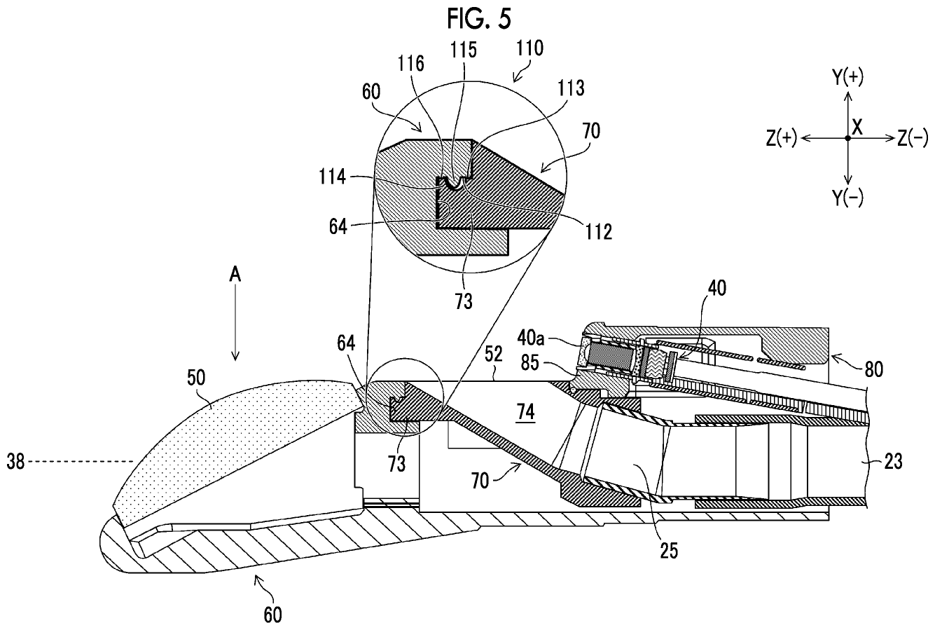

FIG. 5 is a sectional view of the distal end hard part and is an enlarged view of the first load receiving structure. The first load receiving structure 110 is configured by supporting a first supported surface 112 provided in the ultrasound block component 60 by a first support surface 113 provided in the channel block component 70. The first support surface 113 is provided at a position facing the first supported surface 112, and the first support surface 113 supports the first supported surface 112, such that the channel block component 70 can receive the load from the ultrasound block component 60.

The ultrasound block component 60 and the channel block component 70 are assembled by engaging the engaged portion 64 of the ultrasound block component 60 and the engagement portion 73 of the channel block component 70 with each other. As the engagement portion 73, as shown in FIG. 5, a protrusion provided at the distal end of the channel block component 70 can be employed. As the engaged portion 64, a hole shape corresponding to a shape of the protrusion can be employed. In the present embodiment, a surface (a surface toward the Y(−) direction) on the Y(+) direction side of the inside of a hole-shaped portion of the engaged portion 64 is the first supported surface 112. A surface (a surface toward the Y(+) direction) on the Y(+) direction side of the engagement portion 73 is the first support surface 113.

In a case where the ultrasonic wave transmitting and receiving surface (ultrasound transducer 50) is pressed against a living body wall surface, a load is applied in a direction indicated by an arrow A in FIG. 5 by reaction force. The first load receiving structure 110 is provided, whereby the channel block component 70 can receive the load applied to the ultrasound block component 60.

In regard to the connection of the engaged portion 64 and the engagement portion 73, as shown in FIG. 5, the engagement portion 73 has a locking claw 114 corresponding to a locking portion protruding to the Y(+) direction side, on the distal end side. The engaged portion 64 has a locking hole 116 corresponding to a locked portion to which the locking claw 114 is locked, inside the hole shape. In a case of engaging the engaged portion 64 and the engagement portion 73, the engagement portion 73 of the channel block component 70 is inserted into the engaged portion 64 of the ultrasound block component 60. In this case, the locking claw 114 of the engagement portion 73 goes over a convex portion 115 provided on a proximal end side of the engaged portion 64 and is fitted into the locking hole 116 (snap-fit structure). As a result, it is possible to restrict sliding of the engaged portion 64 and of the engagement portion 73, and to restrict movement in the Z direction of the ultrasound block component 60 and of the channel block component 70. In FIG. 5, although the locking claw 114 is provided in the engagement portion 73 and the locking hole 116 is provided in the engaged portion 64, the present invention is not limited to this combination, and a locking hole may be provided in the engagement portion 73 and a locking claw may be provided in the engaged portion 64.

(Second Load Receiving Structure)

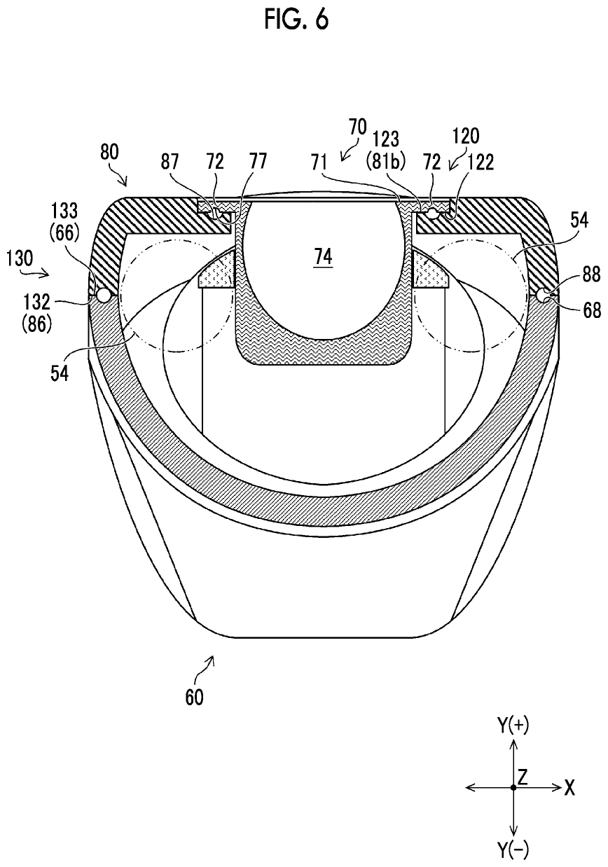

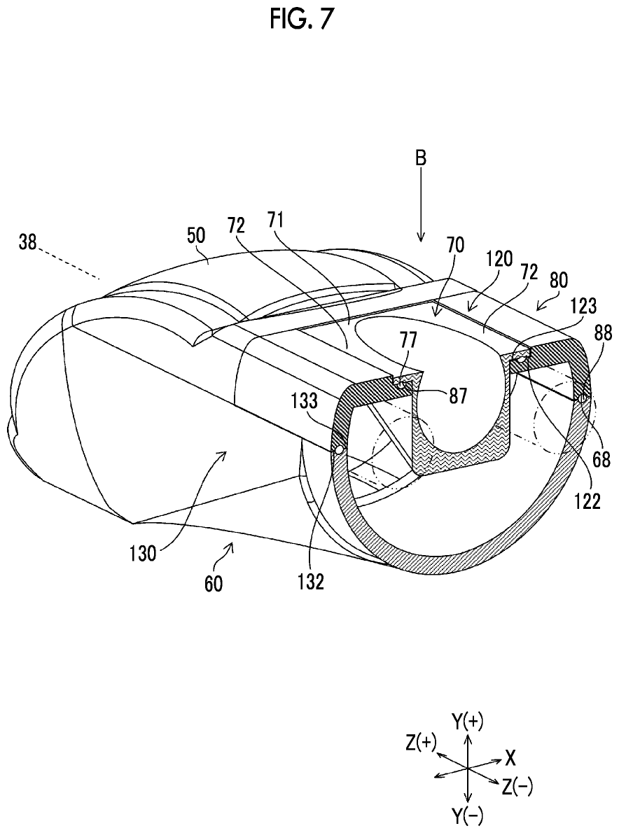

FIG. 6 is a sectional view of the distal end hard part taken along line VI-VI of FIG. 2. FIG. 7 is a perspective view from a cross section side taken along the line VI-VI of FIG. 2. The second load receiving structure 120 is configured by supporting a second supported surface 122 provided in the channel block component 70 by a second support surface 123 provided in the optical system block component 80. The second support surface 123 is provided at a position facing the second supported surface 122, and the second support surface 123 supports the second supported surface 122, whereby the optical system block component 80 can receive the load from the channel block component 70.

As described above, in the channel block component 70, the pair of flange surfaces 72 is formed in both end portions in the X direction of the opening forming surface 71. The pair of flange surfaces 72 is supported by the pair of support surfaces 81b provided in the optical system block component 80, whereby the channel block component 70 is supported by the optical system block component 80. In the present embodiment, a surface on the Y(−) direction side of each of the pair of flange surfaces 72 is the second supported surface 122. A surface on the Y(+) direction side of each of the pair of support surfaces 81b is the second support surface 123.

In a case where the treatment tool (puncture needle) led out from the outlet port 52 is inserted into a living body wall surface, a load is applied in a direction indicated by an arrow B of FIG. 7 by reaction force. The second load receiving structure 120 is provided, whereby the optical system block component 80 can receive the load applied to the channel block component 70.

(Third Load Receiving Structure)

As shown in FIGS. 6 and 7, the third load receiving structure 130 is configured by supporting a third supported surface 132 provided in the optical system block component 80 by a third support surface 133 provided in the ultrasound block component 60. The third support surface 133 is provided at a position facing the third supported surface 132, and the third support surface 133 supports the third supported surface 132, whereby the ultrasound block component 60 can receive the load from the optical system block component 80.

In regard to the attachment of the optical system block component 80 and the ultrasound block component 60, the pair of guided portions 86 of the optical system storage portion 82 of the optical system block component 80 is supported by the pair of guide portions 66 of the optical system block component attachment part 62 of the ultrasound block component 60, whereby the optical system block component 80 is supported by the ultrasound block component 60. In the present embodiment, a surface in the Y(−) direction of each of the pair of guided portions 86 of the optical system block component 80 is the third supported surface 132. A surface in the Y(+) direction of each of the pair of guide portions 66 of the ultrasound block component 60 is the third support surface 133.

With the third load receiving structure 130, the ultrasound block component 60 can receive the load applied to the optical system block component 80.

In this way, with the endoscope of the present embodiment, since the first load receiving structure 110, the second load receiving structure 120, and the third load receiving structure 130 are provided, and each block component of the ultrasound block component 60, the channel block component 70, and the optical system block component 80 can be supported by another block component, it is possible to disperse a load received by any block component to three block components.

In a case where the ultrasonic wave transmitting and receiving surface (ultrasound transducer 50) is pressed against the living body wall surface, reaction force (load) is applied to the ultrasound block component 60. The load received by the ultrasound block component 60 is applied to the channel block component 70 through the first load receiving structure 110. The load received by the channel block component 70 is applied to the optical system block component 80 through the second load receiving structure 120. In this way, since the load received by the ultrasound block component 60 is dispersed to another block component through each load receiving structure, it is possible to improve the strength and durability of the distal end hard part 34.

In a case where the treatment tool (puncture needle) led out from the outlet port 52 is inserted into the living body wall surface, reaction force (load) is applied to the channel block component 70. The load received by the channel block component 70 is applied to the optical system block component 80 through the second load receiving structure 120. The load received by the optical system block component 80 is applied to the ultrasound block component 60 through the third load receiving structure 130. In this case, since the load received by the channel block component 70 is also dispersed to another block component through each load receiving structure, it is possible to improve the strength and durability of the distal end hard part 34.

In the above description, although the reaction force from the living body wall surface applied to the ultrasound block component 60 and the reaction force in a case where the treatment tool is inserted into the living body wall surface have been described, the load applied to the distal end part is not limited thereto. In regard to the load applied to the distal end part, it is possible to disperse the load applied to the optical system block component 80, in addition to the ultrasound block component 60 or the channel block component 70.

<Assembling Method of Endoscope>

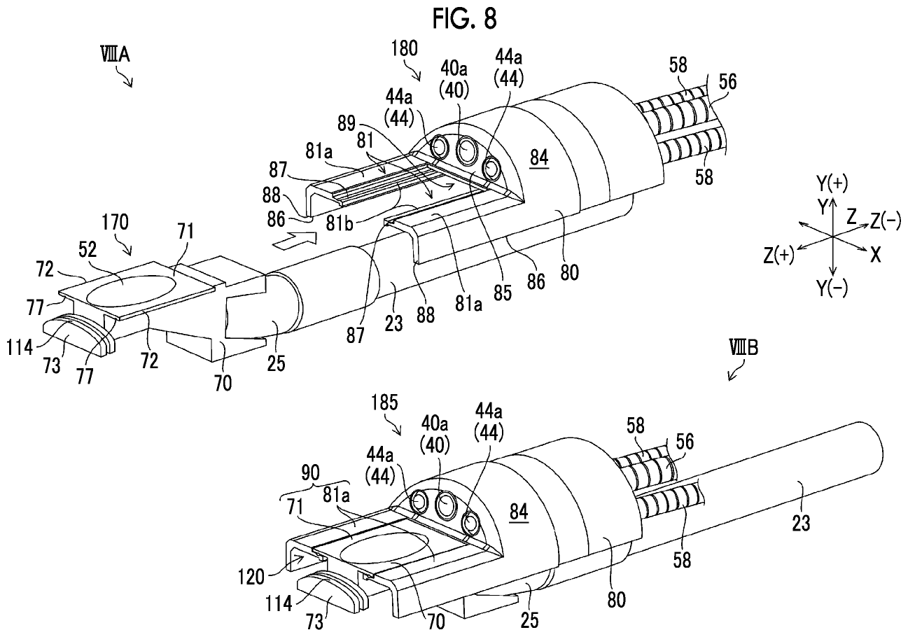

Next, an assembling method of the endoscope will be described. FIGS. 8 to 10 are diagrams illustrating an assembling method of the endoscope.

In assembling the distal end part of the endoscope, first, an optical system sub-assembly 180 and a channel sub-assembly 170 shown in VIIIA of FIG. 8 are formed. The optical system sub-assembly 180 is a component in which the observation optical system 40 and the illumination optical system 44 are assembled to the optical system block component 80. The channel sub-assembly 170 is a component in which the channel connection pipe 25 and the treatment tool insertion channel 23 are assembled to the channel block component 70.

Next, the optical system sub-assembly 180 and the channel sub-assembly 170 are assembled. In assembling the optical system sub-assembly 180 and the channel sub-assembly 170, the pair of flange surfaces 72 (second supported surface 122) provided in the channel block component 70 are slid along the pair of support surfaces 81b (second support surface 123) formed in the optical system block component 80 from the distal end side of the optical system block component 80, and are attached to the channel block component attachment portions 81 to form a channel-optical system sub-assembly 185 (VIIIB of FIG. 8). The pair of support surfaces 81b corresponds to a first guide portion along which the flange surfaces 72 of the channel block component 70 are slidable and disposable. The channel sub-assembly 170 is attached to the channel block component attachment portion 81, whereby the second load receiving structure 120 is formed (second load receiving structure forming step). It is preferable that the optical system sub-assembly 180 and the channel sub-assembly 170 are fixed by an adhesive by applying an adhesive to the groove portions 77 and 87 for an adhesive to ensure strength in the portions of the pair of flange surfaces 72 and the pair of support surfaces 81b.

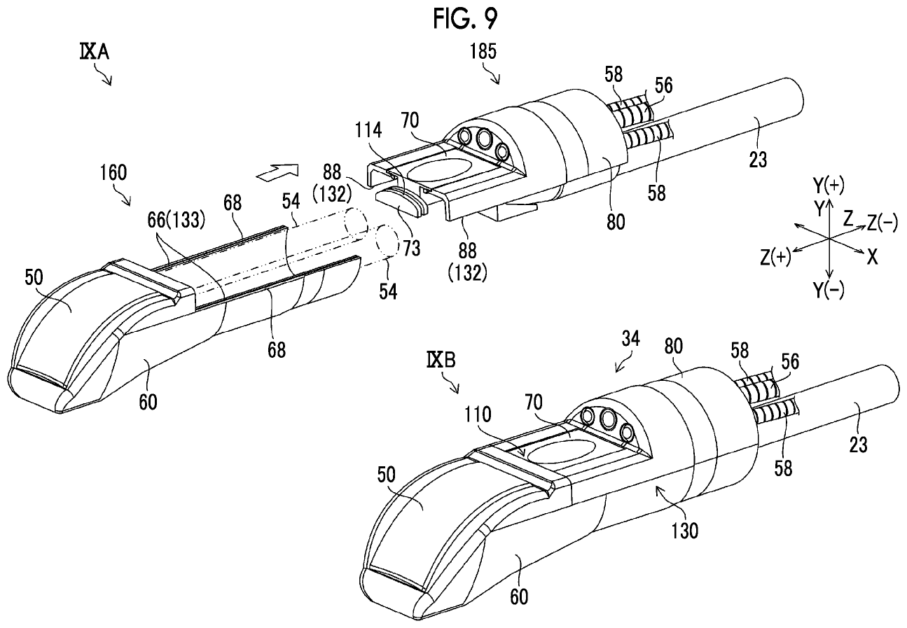

Next, an ultrasound sub-assembly 160 shown in IXA of FIG. 9 in which the ultrasound transducer 50 and the signal cables 54 are assembled to the ultrasound block component 60 is formed.

Then, the channel-optical system sub-assembly 185 and the ultrasound sub-assembly 160 are assembled. The optical system sub-assembly 180 and the ultrasound sub-assembly 160 are assembled by sliding the pair of guided portions 86 of the optical system block component 80 along the pair of guide portions 66 of the ultrasound block component 60 in the Z direction. The pair of guide portions 66 of the ultrasound block component 60 corresponds to a second guide portion along which the optical system block component 80 is slidable and disposable.

It is preferable that the ultrasound block component 60 and the optical system block component 80 are assembled by filling the groove portions 68 and 88 provided in the pair of guide portions 66 of the ultrasound block component 60 and the pair of guided portions 86 of the optical system block component 80 with a seal material to secure airtightness of the inside of the distal end hard part 34.

The optical system sub-assembly 180 to which the channel sub-assembly 170 is attached is slid along the guide portions 66 of the ultrasound block component 60, whereby the engagement portion 73 provided in the channel block component 70 is engaged with the engaged portion 64 provided in the ultrasound block component 60 (see FIG. 5). As a result, the first load receiving structure 110 is formed (first load receiving structure forming step).

The engagement portion 73 and the engaged portion 64 are engaged, whereby the channel-optical system sub-assembly 185 is assembled to the ultrasound block component 60 (IXB of FIG. 9). As a result, the pair of guided portions 86 (third supported surface 132) of the optical system block component 80 is supported by the pair of guide portions 66 (third support surface 133) of the ultrasound block component 60, and the third load receiving structure 130 is formed (third load receiving structure forming step).

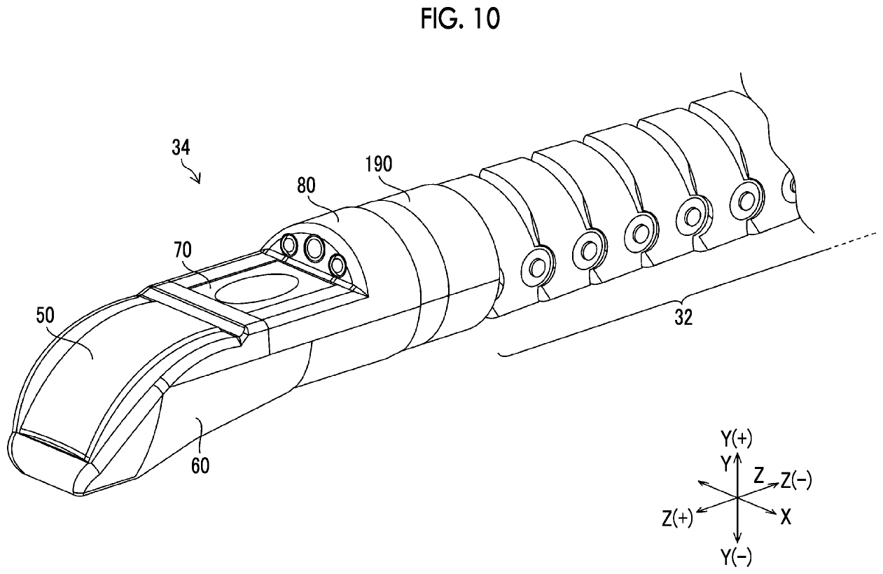

Finally, as shown in FIG. 10, in a state in which the ultrasound block component 60 and the optical system block component 80 are assembled, the outer peripheral surface on the proximal end side is externally fitted and fixed by a bendable ring 190 on the distal end side of the bendable part 32. As a result, the optical system block component 80 and the ultrasound block component 60 are held to be inseparable in the Y direction, and the optical system block component 80 is assembled to the ultrasound block component 60.

The distal end hard part 34 is assembled by such a method, whereby it is possible to easily disassemble the ultrasound sub-assembly 160 and the channel-optical system sub-assembly 185 since the ultrasound sub-assembly 160 and the channel-optical system sub-assembly 185 in which the channel sub-assembly 170 and the optical system sub-assembly 180 are combined are connected by a seal material and are externally fitted and fixed by the bendable ring 190. The distal end hard part 34 as the distal end part of the insertion part 12 is disassembled by the ultrasound sub-assembly 160 and the channel-optical system sub-assembly 185. Accordingly, in a case where a component is broken, since it is possible to only replace the broken component, repair costs can be reduced.

### Another Embodiment

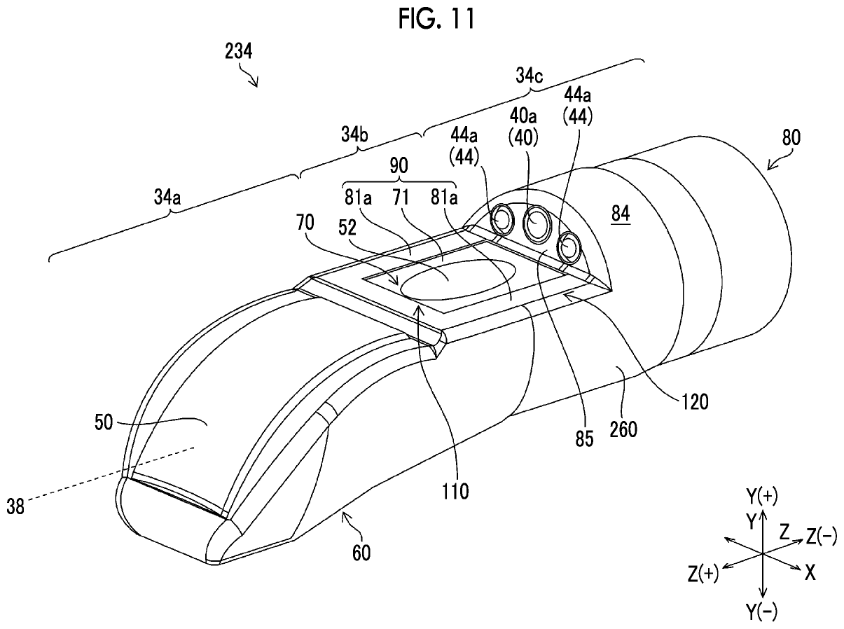

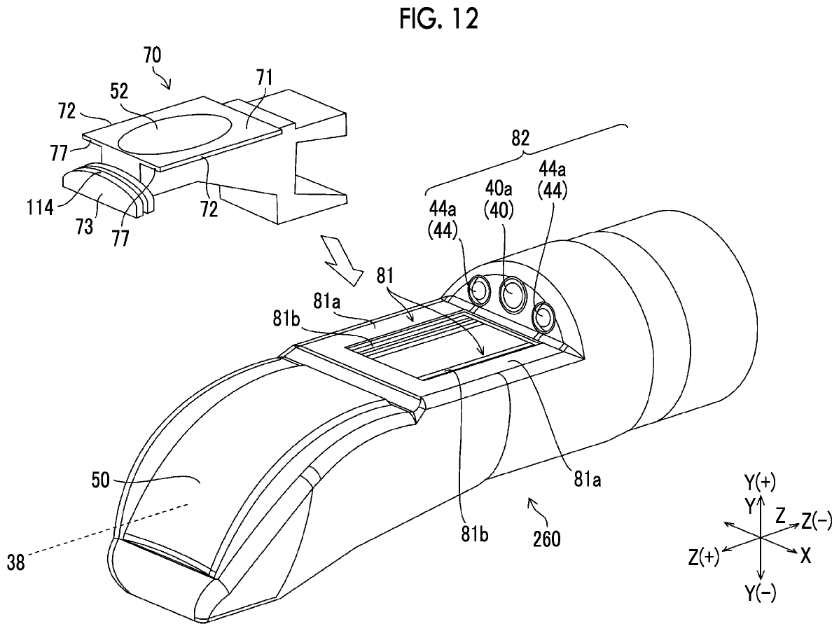

FIG. 11 is a perspective view of a distal end hard part of another embodiment. FIG. 12 is an exploded perspective view of the distal end hard part.

A distal end hard part 234 shown in FIG. 11 is different from the distal end hard part 34 in that two block components are combined: a distal end body block component 260 in which the ultrasound block component 60 of the distal end hard part 34 and the optical system block component 80 of the embodiment described above are integrated, and the channel block component 70.

Also in the distal end hard part 234 of this other embodiment, an engagement portion 73 that is engaged with the distal end body block component 260 is provided on the distal end side of the channel block component 70. An engaged portion (not shown) with which the engagement portion 73 of the channel block component 70 is engaged is formed in the proximal end part of the ultrasonic attachment part 34a of the distal end body block component 260. The engagement portion 73 is engaged with the engaged portion, whereby the first load receiving structure 110 is provided.

The pair of flange surfaces 72 formed in the channel block component 70 is supported by the pair of support surfaces 81b formed in the distal end body block component 260, whereby the second load receiving structure 120 is provided.

In this way, the distal end hard part 234 is configured with the two block components of the distal end body block component 260 in which the ultrasound transducer 50, the observation optical system 40, and the illumination optical system 44 are mounted, and the channel block component 70. The first load receiving structure 110 and the second load receiving structure 120 that receive the loads applied to the respective block components are provided, whereby it is possible to disperse the loads to the respective block components.

In a case where the ultrasonic wave transmitting and receiving surface (ultrasound transducer 50) is pressed against the living body wall surface, the reaction force (load) is applied to the distal end body block component 260. The load received by the distal end body block component 260 is applied to the channel block component 70 through the first load receiving structure 110. The load received by the channel block component 70 is applied to the distal end body block component 260 through the second load receiving structure 120. In this case, the load received by the distal end body block component 260 is applied to the distal end body block component 260 through the first load receiving structure 110, the channel block component 70, and the second load receiving structure 120, and the load received by the distal end body block component 260 can be dispersed to each block component.

In a case where the treatment tool (puncture needle) led out from the outlet port 52 is inserted into the living body wall surface, the reaction force (load) is applied to the channel block component 70. The load received by the channel block component 70 is applied to the distal end body block component 260 through the second load receiving structure 120. The load received by the distal end body block component 260 is applied to the channel block component 70 through the first load receiving structure 110. In this case, the load received by the channel block component 70 is also applied to the channel block component 70 through the second load receiving structure 120, the distal end body block component 260, and the first load receiving structure 110, and the load received by the channel block component 70 can be dispersed to each block component.

In this case, since the load received by one block component can be dispersed to another block component through each load receiving structure, it is possible to improve the strength and durability of the distal end hard part 234.

### EXPLANATION OF REFERENCES

1: ultrasonic endoscope (endoscope)
10: operating part
12: insertion part
14: universal cord
16: angle lever
22: suction button
23: treatment tool insertion channel
24: treatment tool inlet port
25: channel connection pipe
30: soft part
32: bendable part
34: distal end hard part
34a: ultrasonic attachment part
34b: outlet port forming part
34c: body part
38: longitudinal axis of distal end hard part (insertion part)
40: observation optical system
40a: observation window
40b: lens system
40c: imaging element
44: illumination optical system
44a: illumination window
50: ultrasound transducer
52: outlet port
54: signal cable
56: signal cable
58: light guide
60: ultrasound block component
62: optical system block component attachment part
64: engaged portion
65: attachment part opening
66: pair of guide portions
68: groove portion
70: channel block component
71: opening forming surface
72: flange surface
73: engagement portion
74: in-block pipe line
77: groove portion for adhesive
80: optical system block component
81: channel block component attachment portion
81a: pair of planes
81b: pair of support surfaces
82: optical system storage portion
84: convex surface
85: stepped surface
86: pair of guided portions
87: groove portion for adhesive
88: groove portion
89: storage opening portion
90: continuous plane
110: first load receiving structure
112: first supported surface
113: first support surface
114: locking claw
115: convex portion
116: locking hole
120: second load receiving structure
122: second supported surface
123: second support surface
130: third load receiving structure
132: third supported surface
133: third support surface
160: ultrasound sub-assembly
170: channel sub-assembly
180: optical system sub-assembly
185: channel-optical system sub-assembly
190: bendable ring
234: distal end hard part
260: distal end body block component

What is claimed is:

1. An ultrasonic endoscope comprising:
an ultrasound transducer in a distal end part;
a distal end body block component in which the ultrasound transducer, an observation optical system, and an illumination optical system are mounted; and
a channel block component in which a channel into which a treatment tool is inserted is mounted,
wherein the ultrasonic endoscope has
a first load receiving structure that has a first supported surface provided in the distal end body block component and a first support surface provided in the channel block component to face the first supported surface, and in which the first support surface supports the first supported surface, such that the channel block component receives a load from the distal end body block component, and
a second load receiving structure that has a second supported surface provided in the channel block component and a second support surface provided in the distal end body block component to face the second supported surface, and in which the second support surface supports the second supported surface, such that the distal end body block component receives a load from the channel block component.

2. The ultrasonic endoscope according to claim 1,
wherein the distal end body block component includes
an ultrasound block component in which the first supported surface is provided and the ultrasound transducer is mounted, and
an optical system block component in which the second support surface is provided and the observation optical system and the illumination optical system are mounted, and
the ultrasonic endoscope has a third load receiving structure that has a third supported surface provided in the optical system block component and a third support surface provided in the ultrasound block component to face the third supported surface, and in which the third support surface supports the third supported surface, such that the ultrasound block component receives a load from the optical system block component.

3. The ultrasonic endoscope according to claim 2,
wherein the third supported surface and the third support surface are perpendicular to a scanning surface of the ultrasound transducer and are parallel to a surface perpendicular to a longitudinal axis direction of the distal end part.

4. The ultrasonic endoscope according to claim 2,
wherein at least one of the third supported surface or the third support surface has a seal material filling groove portion.

5. The ultrasonic endoscope according to claim 2,
wherein the optical system block component has a first guide portion along which the channel block component is slidable and disposable.

6. The ultrasonic endoscope according to claim 2,
wherein the ultrasound block component has a second guide portion along which the optical system block component is slidable and disposable.

7. The ultrasonic endoscope according to claim 1,
wherein a forming material of the distal end body block component is resin, and
a forming material of the channel block component is metal.

8. The ultrasonic endoscope according to claim 1,
wherein the second supported surface is configured with a pair of flange surfaces that spread outward from both side surfaces of the channel block component opposite to each other.

9. The ultrasonic endoscope according to claim 1,
wherein the distal end body block component has an engaged portion in which the first supported surface is provided, and
the channel block component has an engagement portion in which the first support surface is provided and which is engageable with the engaged portion, and the engaged portion and the engagement portion are engaged with each other, such that the channel block component is assembled to the distal end body block component.

10. The ultrasonic endoscope according to claim 9,
wherein a locking portion is provided in any one of the engaged portion or the engagement portion, and a locked portion that is locked to the locking portion to restrict sliding of the engagement portion with respect to the engaged portion is provided in the other portion.

11. A method for assembling an ultrasonic endoscope including an ultrasound transducer in a distal end part, the method comprising:
a first load receiving structure forming step of forming a first load receiving structure having a structure in which a distal end body block component in which the ultrasound transducer, an observation optical system, and an illumination optical system are mounted is supported by a channel block component in which a channel into which a treatment tool is inserted is mounted; and
a second load receiving structure forming step of forming a second load receiving structure having a structure in which the channel block component is supported by the distal end body block component.

12. The method for assembling an ultrasonic endoscope according to claim 11,
wherein the distal end body block component includes an ultrasound block component in which the ultrasound transducer is mounted, and an optical system block component in which the observation optical system and the illumination optical system are mounted,
the first load receiving structure is a structure in which the ultrasound block component is supported by the channel block component,
the second load receiving structure is a structure in which the channel block component is supported by the optical system block component, and
the method for assembling an ultrasonic endoscope further comprises:
a third load receiving structure forming step of forming a third load receiving structure having a structure in which the optical system block component is supported by the ultrasound block component.

13. The method for assembling an ultrasonic endoscope according to claim 12,
wherein the first load receiving structure forming step and the third load receiving structure forming step are performed after the second load receiving structure forming step is performed.
